(19) United States
(12) Patent Application Publication
(10) Pub. No.: US 2005/0058606 A1
(43) Pub. Date: Mar. 17, 2005

(54) Tiotropium containing HFC solution formulations

(75) Inventors: Sabine Six, Wiesbaden (DE); Christel Schmelzer, Ingelheim (DE); Friedrich Schmidt, Ingelheim (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim Pharma GmbH & Co. KG, Ingelheim (DE)

(21) Appl. No.: 10/730,796

(22) Filed: Dec. 9, 2003

Related U.S. Application Data
(60) Provisional application No. 60/446,672, filed on Feb. 11, 2003.

(30) Foreign Application Priority Data
Dec. 16, 2002 (EP) EP 02 028 238

(51) Int. Cl. A61K 9/14; A61L 9/04; A61K 31/4745

(52) U.S. Cl. 424/46; 514/291

(57) ABSTRACT

This invention relates to tiotropium containing stable pharmaceutical solution formulations suitable for aerosol administration. More particularly, this invention relates to tiotropium containing stable pharmaceutical solution formulations suitable for aerosol administration wherein either an inorganic acid or an organic acid is added to the aerosol solution formulation which contains a tiotropium salt, preferably tiotropium bromide in solution with an environmentally safe hydrofluorocarbon (HFC) as a propellant, together with an organic compound as a cosolvent. The acid provides stability against degradation or decomposition of the medicament resulting largely from interaction of the medicament with the cosolvent and/or water present in the solution formulation.

### RELATED APPLICATIONS

Benefit of U.S. Provisional Application Ser. No. 60/446,672, filed on Feb. 11, 2003 is hereby claimed.

### FIELD OF THE INVENTION

This invention relates to tiotropium containing stable pharmaceutical solution formulations suitable for aerosol administration. More particularly, this invention relates to tiotropium containing stable pharmaceutical solution formulations suitable for aerosol administration wherein either an inorganic acid or an organic acid is added to the aerosol solution formulation which contains a tiotropium salt, preferably tiotropium bromide in solution with an environmentally safe hydrofluorocarbon (HFC) as a propellant, together with an organic compound as a cosolvent. The acid provides stability against degradation or decomposition of the medicament resulting largely from interaction of the medicament with the cosolvent and/or water present in the solution formulation.

### BACKGROUND OF THE INVENTION

Tiotropium bromide is known from European Patent Application EP 418 716 A1 and has the following chemical structure:

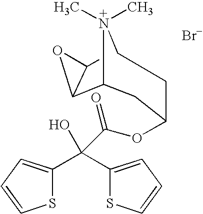

Tiotropium bromide is a highly effective anticholinergic with a long-lasting activity which can be used to treat respiratory complaints, particularly COPD (chronic obstructive pulmonary disease) and asthma. The term tiotropium refers to the free ammonium cation.

For treating the abovementioned complaints, it is useful to administer the active substance by inhalation. In addition to the administration of broncholytically active compounds in the form of inhalable powders containing the active substance the administration of tiotropium bromide can also occur in form of hydrofluorocarbon containing aerosol solution formulations.

The administration of aerosol formulations of medicaments by means of pressurized, metered-dose inhalers (MDIs) is used widely in therapy, such as in the treatment of obstructive airway diseases and asthma. Compared with oral administration, inhalation provides more rapid onset of action while minimizing systemic side effects. Aerosol formulations can be administered by inhalation through the mouth or topically by application to the nasal mucosa.

Formulations for aerosol administration via MDIs can be solutions or suspensions. Solution formulations offer the advantage of being homogeneous in nature with the medicament and excipient completely dissolved in the propellant vehicle. Solution formulations also obviate physical stability problems associated with suspension formulations and thus assure more consistent uniform dosage administration while also eliminating the need for surfactants.

The administration of aerosol solution formulations via MDIs is dependent upon the propulsive force of the propellant system used in its manufacture. Traditionally, the propellant comprised a mixture of chlorofluorocarbons (CFCs) to provide the desired solubility, vapor pressure, and stability of the formulation. However, since it has been established in recent years that CFCs are environmentally harmful because they contribute to the depletion of the Earth's ozone layer, it is desirable to substitute environmentally safe hydrofluorocarbon (HFC) propellants or other non-chlorinated propellants for environmentally harmful CFC propellants in aerosol inhalation formulations. For example, U.S. Pat. No. 4,174,295 discloses the use of propellant systems consisting of combinations of HFCs, which may also contain a saturated hydrocarbon component, suitable for application in the fields of home products such as hair lacquers, anti-perspiration products, perfumes, deodorants, paints, insecticides and the like.

It is known in the art that certain HFCs have properties suitable for use as propellants for the aerosol administration of medicaments. For example, published European patent Application No. 0 372 777 (EPO89312270.5) describes the use of 1,1,1,2-tetrafluoroethane (HFC-134(a)) in combination with at least one “adjuvant” (a compound having a higher polarity than the HFC-134(a)) and a surface active agent to prepare suspension and solution formulations of medicaments suitable for administration by the aerosol route. Also, PCT Published Application No. WO91/11496 (PCT/EP91/00178) discloses the use of 1,1,1,2,3,3,3-heptafluoropropane (HFC-227), optionally mixed with other propellant components, for use in preparing suspension aerosol formulations of medicaments.

U.S. Pat. No. 2,868,641 and U.S. Pat. No. 3,282,781 disclose aerosol compositions comprising a medicament (epinephrine or isoproterenol HCl), a cosolvent, a propellant and ascorbic acid as anti-oxidant. European Patent EP 673 240 B1 proposes the addition of acids to medicinal aerosol formulations in order to provide for the stabilization of the medicament.

### DESCRIPTION OF THE INVENTION

The term “aerosol solution formulation” means a pharmaceutical formulation of a medicament suitable for aerosol administration wherein the medicament and excipients are completely dissolved.

The term “stabilized aerosol solution formulation” means an aerosol solution formulation which exhibits substantial chemical stability over time.

The present invention provides stabilized aerosol solution formulations comprising a tiotropium salt, an HFC propellant, a cosolvent, and an inorganic or an organic acid, characterized in that the concentration of the acid is in a range that corresponds with a pH range of 2.5-4.5 in aqueous solution.

In preferred aerosol solution formulations according to the invention the concentration of the acid is in a range that corresponds with a pH range of 3.0-4.3, more preferred 3.5-4.0 in aqueous solution.

A small amount of water (up to about 5%, preferably up to about 3% by weight,) may also be present in the propellant/cosolvent system.

The aerosol solution formulation according to the invention preferably contains 0.00008 to 0.4%, preferably 0.0004 to 0.16%, more preferably 0.0008 to 0.08% tiotropium. By tiotropium is meant the free ammonium cation. In the tiotropium salt present in the formulation according to the invention the counter-ion (anion) may be chloride, bromide, iodide, methanesulphonate or para-toluenesulphonate. Of these anions, the bromide is preferred.

If the preferred tiotropium salt tiotropium bromide is used, the aforementioned amounts correspond to 0.000096 to 0.48% tiotropium bromide, preferably 0.00048 to 0.192%, more preferably 0.00096 to 0.096% tiotropium bromide.

Tiotropium bromide is, depending on the choice of reaction conditions and solvents, obtainable in different crystalline modifications. Most preferred according to the invention are those formulations, that contain tiotropium in form of the tiotropium bromide monohydrate as disclosed in WO 02/30928. This tiotropium bromide monohydrate is characterised by an endothermic peak at 230±5° C. as determined by DSC.

Accordingly, the aerosol solution formulations according to the invention preferably contains 0.0001 to 0.5% tiotropium bromide monohydrate, preferably 0.0005 to 0.2%, more preferably 0.001 to 0.1% tiotropium bromide monohydrate.

Suitable HFC propellants are those which, when mixed with the cosolvent(s), form a homogeneous propellant system in which a therapeutically effective amount of the medicament can be dissolved. The HFC propellant must be toxicologically safe and must have a vapor pressure which is suitable to enable the medicament to be administered via a pressurized MDI. Additionally, the HFC propellant must be compatible with the components of the MDI device (such as containers, valves, and sealing gaskets, etc.) which is employed to administer the medicament. Preferred HFC propellants are 1,1,1,2-tetrafluoroethane (HFC-134(a)) and 1,1,1,2,3,3,3,-heptafluoropropane (HFC-227). HFC-134(a) is particularly preferred. Other examples of HFC propellants are HFC-32 (difluoromethane), HFC-143(a) (1,1,1-trifluoroethane), HFC-134 (1,1,2,2-tetrafluoroethane), and HFC-152a (1,1-difluoroethane).

It will be apparent to those skilled in the art that non-halogenated hydrocarbon propellants may be used in place of the HFC propellants in the present invention. Examples of non-halogenated hydrocarbons are saturated hydrocarbons, including propane, n-butane, and isobutane, and ethers, including diethyl ether.

It will also be apparent to those skilled in the art that, although the use of a single HFC propellant is preferred, a mixture of two or more HFC propellants, or a mixture of at least one HFC propellant and one or more non-CFC propellants, may be employed in the aerosol solution formulation of the present invention.

A substantially non-aqueous HFC propellant/cosolvent system is preferred. Water may be present in small amounts as an impurity in the HFC propellant/cosolvent system, may be introduced during the manufacturing process or may permeate into the system through the valve or valve/container seals or gaskets. If desired, small amounts of water may be added (up to about 5%, preferably up to about 2% by weight) to the HFC/propellant system, for example, to aid in manufacturing.

The acid in the formulations according to the invention may be any inorganic or mineral acid, for example, hydrochloric acid, sulfuric acid, nitric acid, or phosphoric acid, or the like. From the aforementioned acids hydrochloric acid is of particular interest. The acid may also be selected from the group of acids known to those skilled in the art as organic acids, which are in most cases considered to be weak acids relative to the inorganic acids. Representative of this group and preferred in this invention are ascorbic acid, citric acid, lactic acid, malic acid, benzoic acid and tartaric acid. According to this invention, citric acid and ascorbic acid are the most preferred organic acids.

The formulations according to the invention can be prepared in analogy to methods known in the art.

If desired, pharmaceutically acceptable excipients can be included in the aerosol solution formulations of the present invention. For example, a soluble surface active agent can be added in order to improve the performance of valve systems employed in the MDI devices used for the aerosol administration of the formulations. Examples of preferred surface active agents are sorbitan trioleate, lecithin, and isopropylmyristate. Other suitable lubricants are well known in the art (see, for example, Published European Patent Application No. 0372777 (EPO 893122705)). Other excipients are: (a) antioxidants, for example ascorbic acid and tocopherol; (b) taste masking agents, for example, menthol, sweeteners, and artificial or natural flavors; and (c) pressure modifying agents, for example, n-pentane, iso-pentane, neo-pentane, and n-hexane.

Examples of cosolvents applicable within the formulations according to the invention are: alcohols, for example, ethyl alcohol, isopropyl alcohol, and benzyl alcohol; glycols for example, propylene glycol, polyethylene glycols, polypropylene glycols, glycol ethers, and block copolymers of oxyethylene and oxypropylene; and other substances, for example, glycerol, polyoxyethylene alcohols, polyoxtethylene fatty acid esters, and glycofurols (for example glycofurol 75).

Examples of cosolvents that may be inert to interaction with the medicament(s) are hydrocarbons, for example, n-propane, n-butane, isobutane, n-pentane, iso-pentane, neo-pentane, and n-hexane; and ethers, for example, diethyl ether.

A preferred cosolvent according to this invention is ethyl alcohol (ethanol).

The amount of cosolvent is preferably in the range of 5-50% (w/w) of the total composition. More preferably, the amount of co-solvent in the formulation according to the invention is in the range of 10-40% (w/w), preferably in the range of 15-30%.

As mentioned hereinbefore the formulations according to the invention may contain water a small amount of water. One preferred embodiment of the invention pertains to formulations that contain water in an amount of up to 5% (w/w), preferably of up to 3% (w/w). Another preferred embodiment of the invention is directed to formulations that do not contain any water. In these water-free formulations the amount of cosolvent is preferably in the range of about 20-50% (w/w), more preferably in the range of about 30-40% (w/w).

Especially in these water-free formulations the anhydrous form of tiotropium bromide obtainable from the tiotropium bromide monohydrate mentioned hereinbefore can be used.

The anhydrous form is obtained from the crystalline tiotropium bromide monohydrate disclosed in WO 02/30928 by careful drying at more than 50° C., preferably at 60-100° C., most preferably at 70-100° C., under reduced pressure, preferably in a high vacuum over a period of 15 minutes to 24 hours, preferably 20 minutes to 12 hours, most preferably 30 minutes to 6 hours. The term “reduced pressure” most preferably refers to a pressure of up to 5×10⁻² bar, preferably 1×10⁻² bar, most preferably 5×10⁻³ bar.

Most preferably, the abovementioned dehydration to form the anhydrate is carried out at about 1×10⁻³ bar or less.

Alternatively to the drying step at elevated temperature under reduced pressure described above, the anhydrous form may also be prepared by storing the crystalline tiotropium bromide monohydrate over a drying agent, preferably over dried silica gel at ambient temperature for a period of 12 to 96 hours, preferably 18 to 72 hours, most preferably at least 24 hours. The anhydrous form thus obtained should be stored more or less dry, depending on the particle size, to preserve its anhydrous state. In the case of coarse crystals of anhydrous tiotropium bromide, which may be prepared for example as described above, storage at <75% r.h. (relative humidity) is sufficient to maintain the anhydrous state. In the micronised state, i.e. when the material has a much larger surface area, water may even be absorbed at lower humidity levels. In order to maintain the anhydrous form in the micronised state, it is therefore advisable to store the anhydrous form of tiotropium bromide over dried silica gel.

The anhydrous form of tiotropium bromide was subjected to X-ray analysis which revealed that the crystalline anhydrous tiotropium bromide is characterised by the elementary cells a=10.4336(2)Å, b=11.3297(3)Å, c=17.6332(4)Å and α=90°, β=105.158(2)° and γ=90° (cell volume=2011.89(8)Å³). The crystalline structure of the anhydrous form of tiotropium bromide can be described as a layered structure. The bromide ions are located between the layers of tiotropium. Further details concerning the determination of the crystalline structure of the said anhydrous form are outlined in the experimental part of this patent application.

Accordingly, a further preferred embodiment of the invention is directed to a stabilized aerosol solution formulation comprising anhydrous tiotropium bromide characterized by the aforementioned parameters, an HFC propellant, a cosolvent, and an inorganic or an organic acid, characterized in that the concentration of the acid is in a range that corresponds with a pH range of 2.5-4.5 in aqueous solution and further characterized in that the formulation is free of water.

The formulations according to the invention can be administered with inhalers known in the art (Metered dose inhalers=MDIs).

In another aspect the invention is directed to the use of an aerosol solution formulation as described hereinbefore for the manufacture of a medicament for the treatment of respiratory complaints, particularly COPD (chronic obstructive pulmonary disease) and asthma.

In yet another aspect the invention is directed to a method for treatment of respiratory complaints, such as in particular COPD (chronic obstructive pulmonary disease) or asthma, characterized by the administration of an aerosol solution formulation as described hereinbefore.

The following Examples serve to illustrate the present invention further without restricting its scope to the embodiments provided hereinafter by way of example.

### I. FORMULATION EXAMPLES

| A) | Component | Concentration [% w/w] |
|---|---|---|
| | Tiotropium bromide monohydrate | 0.02 |
| | Ethanol abs. (USP) | 25.0 |
| | Water (purified, USP) | 1.0 |
| | Citric acid (USP) | 0.003 |
| | HFC-134a | 73.977 |

| B) | Component | Concentration [% w/w] |
|---|---|---|
| | Tiotropium bromide monohydrate | 0.02 |
| | Ethanol abs. (USP) | 20.0 |
| | Aqueous HCl 0.01 mol/l (USP) | 2.0 |
| | HFC-134a | 77.98 |

| C) | Component | Concentration [% w/w] |
|---|---|---|
| | Tiotropium bromide monohydrate | 0.01 |
| | Ethanol abs. (USP) | 15.0 |
| | Water (purified, USP) | 2.0 |
| | Citric acid (USP) | 0.004 |
| | HFC-227 | 82.986 |

| D) | Component | Concentration [% w/w] |
|---|---|---|
| | Tiotropium bromide monohydrate | 0.01 |
| | Ethanol abs. (USP) | 30.0 |
| | Water (purified, USP) | 1.0 |
| | Ascorbic acid (USP) | 0.005 |
| | HFC-134a | 68.985 |

| E) | Component | Concentration [% w/w] |
|---|---|---|
| | Tiotropium bromide (anhydrous) | 0.01 |
| | Ethanol abs. (USP) | 40.0 |
| | citric acid (USP) | 0.004 |
| | HFC-227 | 59.986 |

The aformentioned formulations can be prepared by conventional methods known in the state of the art.

II. Preparation of Crystalline Anhydrous Tiotropium Bromide:

The anhydrous form is produced from the crystalline tiotropium bromide monohydrate (obtainable as described in WO 02/30928) by careful drying at 80-100° C. under reduced pressure, preferably under a high vacuum (at about 1×10⁻³ bar or less) over a period of at least 30 minutes. Alternatively to the drying step at 80-100° C. in vacuo the anhydrous form may also be prepared by storing over dried silica gel at ambient temperature for a period of at least 24 hours.

III. Characterisation of Crystalline, Anhydrous Tiotropium Bromide

As described hereinbefore, the crystalline anhydrous tiotropium bromide according to the invention may be obtained from crystalline tiotropium bromide monohydrate.

The crystalline structure of anhydrous tiotropium bromide was determined from high-resolution X-ray powder data (synchrotron radiation) using a real space approach with a so-called simulated annealing process. A final Rietveld analysis was carried out to refine the structural parameters. Table 1 contains the experimental data obtained for crystalline, anhydrous tiotropium bromide.

TABLE 1
Experimental data relating to the crystalline structural
analysis of tiotropium bromide (anhydrous)

| formula | C₁₉H₂₂NO₄S₂Br |
|---|---|
| temperature [° C.] | 25 |
| molecular weight [g/mol] | 472.4 |
| space group | P2₁/c |
| a [Å] | 10.4336(2) |
| b [Å] | 11.3297(3) |
| c [Å] | 17.6332(4) |
| β[°] | 105.158(2) |
| V [Å₃] | 2011.89(8) |
| Z | 4 |
| calculated density [g cm⁻³] | 1.56 |
| 2Θ (range) [°] | 2.0-20 |
| interval [°2Θ] | 0.003 |
| counting time/step [sec] | 3 |
| wavelength [Å] | 0.7000 |

The crystalline structure of the anhydrous form of tiotropium bromide can be described as a layered structure. The bromide ions are located between the layers of tiotropium.

In order to clarify the structure of crystalline anhydrous tiotropium bromide a high-resolution X-ray powder diagram was taken at ambient temperature at the National Synchrotron Source (Brookhaven National Laboratory, USA) at measuring station X3B1 (λ=0.700 Å). For this experiment a sample of crystalline tiotropium bromide monohydrate was placed in a quartz glass capillary 0.7 mm in diameter. The water was eliminated by heating to 80° C. in an oven under reduced pressure.

The structural resolution was obtained by a so-called simulated annealing process. The DASH program package produced by Cambridge Crystallographic Data Center (CCDC, Cambridge, United Kingdom) was used for this.

TABLE 2
Coordinates

| Atom | x | y | z | Uᵢₛₒ |
|---|---|---|---|---|
| S1 | 1.0951(8) | 0.3648(8) | 0.8189(5) | 0.075(9) |
| S1 | 0.9143(9) | 0.1374(8) | 0.9856(5) | 0.075(9) |
| O | 0.6852(13) | 0.2339(6) | 0.7369(6) | 0.075(9) |
| O1 | 0.7389(15) | 0.0898(9) | 0.8234(6) | 0.075(9) |
| O2 | 0.8211(10) | 0.3897(17) | 0.8277(7) | 0.075(9) |
| O3 | 0.4975(17) | 0.4816(9) | 0.6011(7) | 0.075(9) |
| N | 0.4025(10) | 0.2781(8) | 0.5511(5) | 0.075(9) |
| C | 0.7509(8) | 0.1885(6) | 0.8038(5) | 0.075(9) |
| C1 | 0.8593(7) | 0.2788(5) | 0.8495(4) | 0.075(9) |
| C2 | 0.9924(9) | 0.2533(6) | 0.8225(6) | 0.075(9) |
| C3 | 0.8884(9) | 0.2664(7) | 0.9382(4) | 0.075(9) |
| C4 | 0.5848(12) | 0.1596(8) | 0.6753(8) | 0.075(9) |
| C5 | 0.4544(13) | 0.1929(14) | 0.6809(8) | 0.075(9) |
| C6 | 0.6156(13) | 0.1810(13) | 0.5973(9) | 0.075(9) |
| C7 | 0.5493(11) | 0.2881(11) | 0.5578(6) | 0.075(9) |
| C8 | 0.5869(12) | 0.3832(11) | 0.6092(7) | 0.075(9) |
| C9 | 0.4947(13) | 0.3902(10) | 0.6575(6) | 0.075(9) |
| C10 | 0.4004(10) | 0.2998(11) | 0.6332(6) | 0.075(9) |
| C11 | 0.3220(13) | 0.3670(13) | 0.4935(6) | 0.075(9) |
| C12 | 0.3450(19) | 0.1643(26) | 0.5211(11) | 0.075(9) |
| C13 | 0.9184(16) | 0.3808(9) | 0.9920(6) | 0.075(9) |
| C14 | 1.0313(16) | 0.1552(15) | 0.8011(15) | 0.075(9) |
| C15 | 0.9515(17) | 0.3374(10) | 0.0501(6) | 0.075(9) |
| C16 | 0.9756(18) | 0.2190(11) | 1.0742(5) | 0.075(9) |
| C17 | 1.1483(22) | 0.1762(18) | 0.7718(24) | 0.075(9) |
| C18 | 1.1860(16) | 0.2800(15) | 0.7768(19) | 0.075(9) |
| BR | 0.4597(4) | 0.8200(15) | 0.61902(25) | 0.042(9) |

Table 2 shows the atomic cooridinates for crystalline anhydrous tiotopium bromide.

In the above Table the “Uᵢₛₒ” values denote the isotropic temperature factors. For example, in single-crystal X-ray structural analysis this corresponds to the u(eq) values.

TABLE 3
Experimental data relating to the crystalline structural
analysis of anhydrous tiotropium bromide

| No. | h | k | l | 2Θ_{obs.} | 2Θ_{calc.} | 2Θ_{obs.} − 2Θ_{calc} |
|---|---|---|---|---|---|---|
| 1 | 1 | 0 | 0 | 8.762 | 8.769 | −0.007 |
| 2 | 0 | 1 | 1 | 9.368 | 9.369 | −0.001 |
| 3 | −1 | 0 | 2 | 11.730 | 11.725 | 0.005 |
| 4 | 0 | 1 | 2 | 12.997 | 13.004 | −0.007 |
| 5 | −1 | 1 | 2 | 14.085 | 14.094 | −0.009 |
| 6 | 1 | 0 | 2 | 15.271 | 15.275 | −0.004 |
| 7 | 0 | 0 | 3 | 15.620 | 15.616 | 0.004 |
| 8 | 0 | 2 | 1 | 16.475 | 16.475 | 0.0 |
| 9 | 1 | 1 | 2 | 17.165 | 17.170 | −0.005 |
| 10 | 2 | 0 | 0 | 17.588 | 17.591 | −0.003 |
| 11 | −1 | 2 | 1 | 18.009 | 18.035 | −0.026 |
| 12 | 1 | 2 | 1 | 19.336 | 19.328 | 0.008 |
| 13 | −2 | 1 | 2 | 19.596 | 19.600 | −0.004 |
| 14 | −1 | 0 | 4 | 20.417 | 20.422 | −0.005 |
| 15 | 0 | 0 | 4 | 20.865 | 20.872 | −0.007 |
| 16 | 2 | 1 | 1 | 21.150 | 21.145 | 0.005 |
| 17 | −2 | 1 | 3 | 21.759 | 21.754 | 0.005 |
| 18 | 0 | 2 | 3 | 22.167 | 22.160 | 0.007 |
| 19 | −1 | 2 | 3 | 22.289 | 22.288 | 0.001 |
| 20 | 2 | 0 | 2 | 22.735 | 22.724 | 0.011 |
| 21 | −2 | 2 | 1 | 23.163 | 23.159 | 0.004 |
| 22 | −2 | 0 | 4 | 23.567 | 23.575 | −0.008 |
| 23 | 2 | 1 | 2 | 24.081 | 24.058 | 0.023 |
| 24 | 1 | 0 | 4 | 24.746 | 24.739 | 0.007 |
| 25 | −1 | 3 | 1 | 25.220 | 25.221 | −0.001 |
| 26 | 1 | 2 | 3 | 25.359 | 25.365 | −0.006 |
| 27 | 0 | 3 | 2 | 25.790 | 25.783 | 0.007 |
| 28 | 1 | 1 | 4 | 25.978 | 25.975 | 0.003 |
| 29 | 0 | 2 | 4 | 26.183 | 26.179 | 0.004 |
| 30 | −1 | 3 | 2 | 26.383 | 26.365 | 0.018 |
| 31 | −1 | 1 | 5 | 26.555 | 26.541 | 0.014 |
| 32 | −3 | 1 | 2 | 27.024 | 27.021 | 0.003 |
| 33 | 3 | 1 | 0 | 27.688 | 27.680 | 0.008 |
| 34 | −3 | 1 | 3 | 28.221 | 28.215 | 0.006 |
| 35 | 3 | 0 | 1 | 28.377 | 28.376 | 0.001 |
| 36 | −3 | 0 | 4 | 29.246 | 29.243 | 0.003 |
| 37 | 3 | 1 | 1 | 29.459 | 29.471 | −0.012 |
| 38 | −1 | 2 | 5 | 29.906 | 29.900 | 0.006 |
| 39 | −3 | 2 | 1 | 30.171 | 30.165 | 0.006 |
| 40 | 0 | 2 | 5 | 30.626 | 30.626 | 0.0 |
| 41 | 1 | 1 | 5 | 30.871 | 30.856 | 0.015 |
| 42 | 0 | 0 | 6 | 31.504 | 31.532 | −0.028 |
| 43 | 2 | 1 | 4 | 31.826 | 31.847 | −0.021 |
| 44 | −2 | 1 | 6 | 32.888 | 32.888 | 0.0 |
| 45 | 1 | 4 | 1 | 33.605 | 33.615 | −0.010 |
| 46 | 3 | 0 | 3 | 34.379 | 34.377 | 0.002 |
| 47 | 1 | 0 | 6 | 35.021 | 35.018 | 0.003 |
| 48 | −4 | 1 | 1 | 35.513 | 35.503 | 0.01 |
| 49 | 1 | 1 | 6 | 35.934 | 35.930 | 0.004 |
| 50 | −1 | 1 | 7 | 36.544 | 36.543 | 0.001 |
| 51 | −4 | 1 | 4 | 37.257 | 37.255 | 0.002 |
| 52 | −4 | 2 | 2 | 37.933 | 37.952 | −0.019 |
| 53 | 4 | 1 | 1 | 38.258 | 38.264 | −0.006 |

Table 3 shows the reflexes (h,k,l indices) of the powder diagram obtained for crystalline anhydrous tiotropium bromide.

1. A composition comprising tiotropium, or a pharmaceutically acceptable salt or hydrate thereof, an HFC propellant, a solvent, and an acid selected from the group consisting of one or more of inorganic and organic acids having a pH range of 2.5-4.5 in aqueous solution.

2. The composition according to claim 1 comprising 0.00008 to 0.4% by weight tiotropium, or a pharmaceutically acceptable salt or hydrate thereof.

3. The composition according to claim 2, wherein the pharmaceutically acceptable salt of tiotropium is selected from the group consisting of one or more of chloride, bromide, iodide, methanesulphonate or para-toluenesulphonate.

4. The composition according to claim 1 wherein the HFC propellant is selected from the group consisting of HFC-134(a), HFC-227, HFC-32, HFC-143(a), HFC-134, HFC-152a, and mixtures thereof.

5. The composition according to claim 1 wherein the acid is selected from the group of inorganic acids consisting of hydrochloric acid, sulfuric acid, nitric acid, and phosphoric acid.

6. The composition according to claim 1 wherein the acid is selected from the group of organic acids consisting of ascorbic acid, citric acid, lactic acid, malic acid, benzoic acid, and tartaric acid.

7. The composition according to claim 1 further comprising water in an amount of up to about 5% by weight.

8. The composition according to claim 1 wherein the solvent is selected from the group consisting of one or more of alcohols, glycols, glycol ethers, block copolymers of oxyethylene and oxypropylene, glycerol, polyoxyethylene alcohols, polyoxtethylene fatty acid esters and glycofurols.

9. The composition according to claim 8 wherein the solvent is present in an amount in the range of 5-50% by weight.

10. The composition according to claim 1 comprising an anhydrous crystalline form of tiotropium bromide.

11. The composition according to claim 1 that is free of water.

12. The composition according to claim 10 that is free of water.

13. The composition according to claim 2 comprising tiotropium bromide monohydrate in a range of from 0.0001% to 0.5% (by weight), ethanol in the range of 5% to 50% (by weight), water up to 5% (by weight), acid in an amount to yield a pH range of 2.5 to 4.5 in aqueous solution, and an HFC propellant.

14. The composition according to claim 10 comprising anhydrous crystalline tiotropium bromide in the range of 0.0001% to 0.5% (by weight), ethanol in the range of 5% to 50% (by weight), water up to 5% (by weight), acid in an amount to yield a pH range of 2.5 to 4.5 in aqueous solution, and an HFC propellant.

15. A device for the administration of aerosol compositions comprising the composition according to claim 1.

16. A device for the administration of aerosol compositions comprising the composition according to claim 10.

17. A device for the administration of aerosol compositions comprising the composition according to claim 13.

18. A device for the administration of aerosol compositions comprising the composition according to claim 14.

19. The device according to claim 15 in the form of a metered-dose inhaler.

20. The composition according to claim 1 in the form of an aerosol solution formulation.
